(19) United States
(12) Patent Application Publication
(10) Pub. No.: US 2006/0246593 A1
(43) Pub. Date: Nov. 2, 2006

(54) Material processing apparatus and method

(76) Inventor: Gavin P. Towler, Barrington, IL (US)

Correspondence Address: HONEY WELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 10/336,937

(22) Filed: Jan. 3, 2003

(51) Int. Cl. G01N 33/00

(52) U.S. Cl. 436/73

(57) ABSTRACT

An apparatus for contacting fluid with material includes a processing device having a feed port and an effluent port for passing fluid through a cartridge having an inlet and an outlet, a cartridge position controller for positioning the cartridge between and removing the cartridge from between the feed port and the effluent port, a first seal for sealing between the feed port and the cartridge inlet, and a second seal for sealing between the effluent port and the cartridge outlet. A method for processing material includes loading material into a cartridge having an inlet and an outlet, positioning the cartridge between a feed port and an effluent port, sealing between the feed port and the cartridge inlet and between the effluent port and the cartridge outlet, flowing fluid through the cartridge and releasing the cartridge from between the feed port and effluent port.

### BACKGROUND OF THE INVENTION

1. Field of the Invention

The present invention relates to an apparatus and method for processing of a material sample, such as a catalyst.

2. Description of the Related Art

Before a material is selected for use in a commercial application, for example catalysts for hydrocarbon reactions in petroleum refining, a great number of materials may be examined for use in the envisioned application. A large number of material compositions may be synthesized, processed and screened while under consideration as candidates.

The traditional approach to the processing of new material has been a sequential one. One new potential material undergoes a process step in a vessel. Upon completion of the process step, the one material is removed from the vessel and a second material is loaded. The process is repeated on the freshly loaded material. The process is repeated sequentially for each of the materials. Not only is this approach drawn out for a single process step, but the time requirement is compounded by multiple process steps which may require multiple process vessels. Overall, processing of a plurality of new material formulations is a lengthy process at best.

One method that has been employed for individual processing steps is to take a combinatorial approach. Combinatorial chemistry has dealt mainly with the synthesis of new compounds. For example, U.S. Pat. No. 5,612,002 and U.S. Pat. No. 5,766,556 teach an apparatus and a method for simultaneous synthesis of multiple compounds. Akporiaye, D. E.; Dahl, I. M.; Karlsson, A.; Wendelbo, R. Angew Chem. Int. Ed. 1998, 37, 9-611 disclose a combinatorial approach to the hydrothermal synthesis of zeolites, see also WO 98/36826.

Combinatorial approaches have also recently been used for the evaluation and screening of catalysts; see for example commonly assigned U.S. Pat. Nos. 6,342,185 and 6,368,865 and U.S. patent applications Ser. Nos. 10/095,395, 10/095,879 and 10/095,934.

A system for the parallel treatment of a plurality of materials is also disclosed in the commonly assigned patent applications having Attorney Docket Numbers 105279 and 105397, filed contemporaneously herewith, the disclosures of which are incorporated herein by reference.

Efforts have been made to expedite processing of a plurality of materials by placing a small amount of each material into a corresponding number of containers and then processing each container. Attempts also have been made to use several containers simultaneously in an array in order to analyze samples. An example of simultaneously using multiple containers is disclosed in U.S. Pat. No. 4,766,082 and in the Argonaut Endeavor apparatus.

The use of multiple containers eliminates the need to load and unload a single container several times. However, the use of multiple containers still has required manual manipulation of each container, which is a slow and clumsy process, particularly when each individual material, and hence each individual container, must undergo a plurality of sequential process steps. Moreover, it may be desired to vary the process steps, which adds further complication and time.

What is needed is an apparatus and method to automate and speed up the processing of materials in order to improve efficiency of the material preparation and examination process.

### BRIEF SUMMARY OF THE INVENTION

The present invention allows for the rapid movement of material samples by using a processing device to position one or more cartridges from a series of cartridges into the processing device for preparation or examination of a material contained within the cartridge. In accordance with the present invention, a novel and improved apparatus is provided for contacting a plurality of solid material samples with fluid in a series of cartridges. The inventive apparatus includes a processing device for passing fluid through an interior volume of a cartridge, the processing device having a feed port and an effluent port and the cartridge having an inlet and an outlet, at least one cartridge position controller for positioning the cartridge between the feed port and the effluent port and for removing the cartridge from between the feed port and the effluent port, a first seal for sealing between the feed port and the cartridge inlet, and a second seal for sealing between the effluent port and the cartridge outlet.

Also in accordance with the present invention, a novel and improved method is provided for processing material. The inventive method includes the steps of loading material to be processed into a cartridge, wherein the cartridge includes an inlet and an outlet, positioning the cartridge between a feed port and an effluent port, sealing between the cartridge inlet and the feed port and between the cartridge outlet and the effluent port, flowing fluid through the cartridge, and releasing the cartridge from between the feed port and the effluent port.

These and other objects, features and advantages are evident from the following description of an embodiment of the present invention, with reference to the accompanying drawings.

### BRIEF DESCRIPTION OF THE SEVERAL VIEWS OF THE DRAWINGS

FIG. 1 is a side sectional view of a processing device shown with a plurality of cartridges and a cartridge position controller.

FIG. 2 is a perspective view of a cartridge, shown holding a material to be processed.

FIG. 3 is an inverted perspective view of the cartridge, showing a support member for supporting the material.

FIG. 4 is a side sectional view of a processing device having an alternative sealing apparatus.

FIG. 5 is a side sectional view of a plurality of processing devices, shown with a plurality of cartridges and a cartridge position controller.

FIG. 6 is a perspective view of a carousel for holding a plurality of cartridges and a plurality of processing devices.

FIG. 7 is a perspective view of a cartridge tray for holding a plurality of cartridges and a plurality of sets of processing devices.

### DETAILED DESCRIPTION OF THE INVENTION

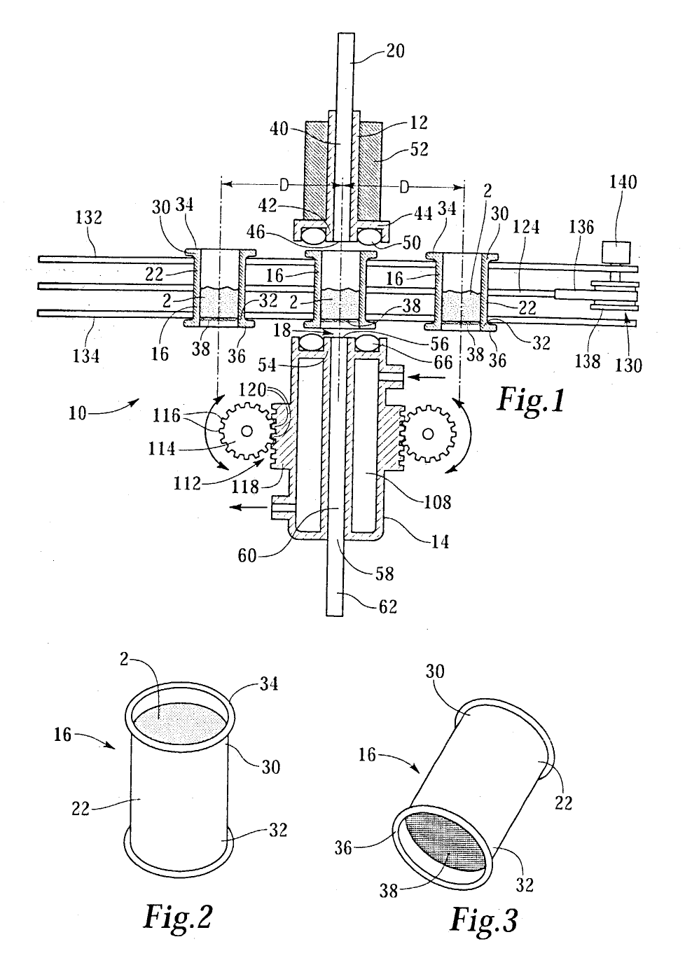

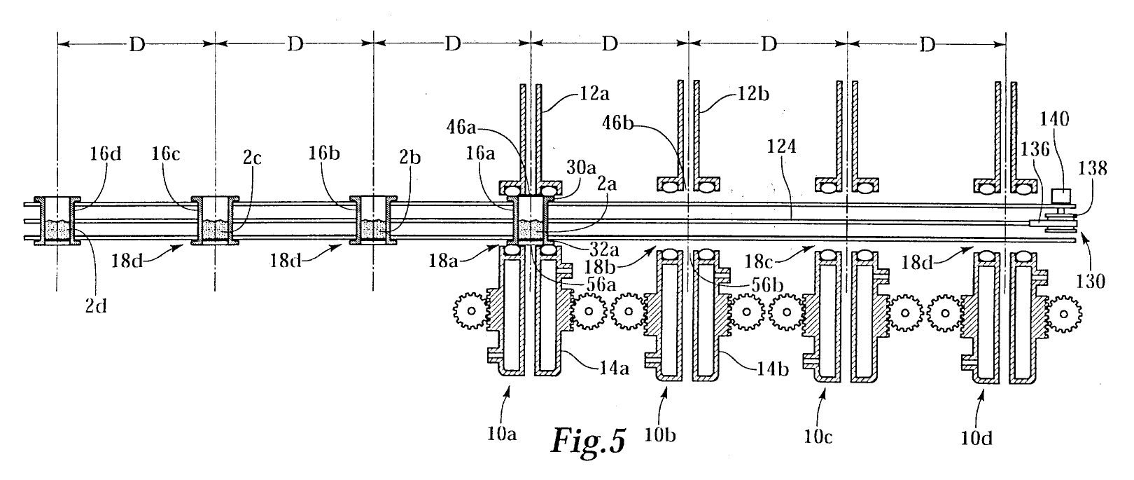

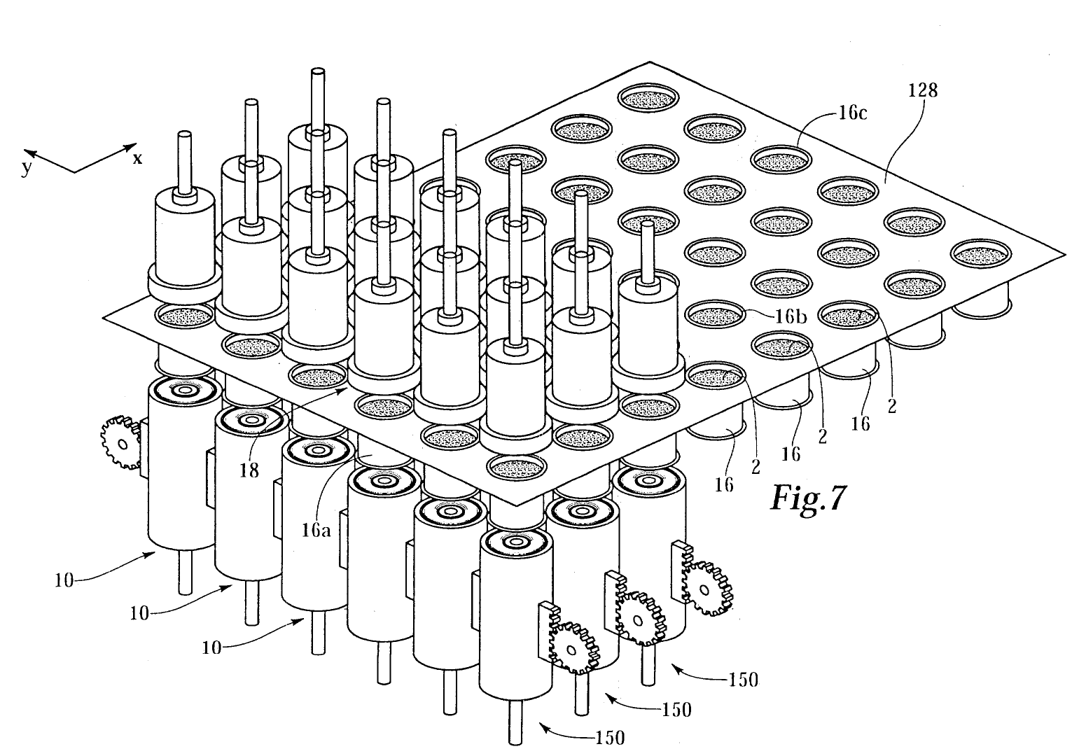

A novel and improved material processing device 10 for processing materials 2 is shown in FIG. 1. An individual processing device 10 or a plurality of processing devices 10a, 10b, 10c, 10d (see FIG. 5) can be used to process one or more materials 2 in series, or a plurality of processing devices 10 can be used in parallel for the simultaneous processing of a plurality of materials 2, as in FIG. 7.

Each processing device 10 includes a feed section 12 having a feed port 46, an effluent section 14 having an effluent port 56 and a cartridge station 18 located between feed section 12 and effluent section 14. A cartridge 16 for holding a small sample of material 2 is placed in cartridge station 18, between feed port 46 and effluent port 56, as is shown in FIG. 1.

Processing device 10 can be used for several types of catalyst or material process steps. Examples of process steps for which process device 10 can be used include preparation steps, pre-treatment steps, treatment steps (such as heat treatment), finishing steps, and screening steps.

A certain set of process conditions define a process step, such as fluid flow rates and composition or temperature. Therefore two operations define different process steps if they process material 2 under different process conditions.

For the example of heat treatment of an inorganic catalyst, in one method processing device 10 can be operated with fluid flow rates between about 0.1 cm³/min to about 1000 cm³/min, preferably between about 0.5 cm³/min and about 25 cm³/min. The fluid can also be heated, as described below, to temperatures between room temperature of about 20° C., to high temperatures of about 1000° C., and preferably between about 300° C. and about 800° C. Other process conditions that can be altered in processing device 10 include materials 2 being processed, and processing fluids used to process materials 2.

Preferred materials 2 that can be processed within processing device 10 include inorganic catalysts, such as metallic catalysts used in the petrochemical industry, metals, and other inorganic materials which may undergo one or more process steps before the material has certain desired properties.

Examples of catalysts that may be processed using the present invention include those effective in a wide variety of hydrocarbon conversion processes such as cracking, hydrocracking, alkylation of both aromatics and isoparaffins, isomerization, polymerization, reforming, dewaxing, hydrogenation, dehydrogenation, transalkylation, dealkylation, hydration, dehydration, hydrotreating, hydrodenitrogenation, hydrodesulfurization, methanation, ring opening, and syn-gas shift processes. Specific examples are discussed in H. Pines, The Chemistry Of Catalytic Hydrocarbon Conversions, Academic Press (1981).

Examples of process fluids being fed to processing device 10 include pure components, such as pure hydrogen gas, oxygen gas, H₂O gas and H₂O liquid, or mixtures of components, such as half nitrogen gas and half air, or a mixture of hydrochloric acid and water (aqueous HCl).

Processing device 10 provides a flow of a process fluid through a sample of material 2 in order to perform a particular process step. The process fluid feeds into feed section 12 through feed line 20. The process fluid then flows through cartridge 16 to process material 2. Eventually the process fluid flows out of cartridge 16 through effluent section 14.

Preferably, process device 10 operates so that cartridge 16 is arranged vertically, as shown in FIG. 1, because it is preferred that the process fluid flow evenly through material 2 as opposed to across material 2, which might process material 2 unevenly, e.g. with more fluid flow across a top layer so that the top layer is processed while a bottom layer remains untreated or vice versa, depending on the relative densities of the fluid and material 2. Even and uniform flowing through material 2 is preferred because it increases the likelihood that a particular process step being performed by processing device 10 is accomplished substantially homogeneously throughout the whole material 2 held in cartridge 16. For example, if processing device 10 is being used for heat treatment of material 2, even flow of a treatment fluid ensures that the substantially the entire sample of material 2 is treated essentially homogeneously by the process fluid.

Turning to FIGS. 1, 2 and 3, a cartridge 16 holds a material 2 to be processed, and in one embodiment is generally cylindrical in shape having a reinforced wall 22 with an inlet end 30 defining an inlet rim 34 and an outlet end 32 defining an outlet rim 36. A support member 38 is connected to cartridge 16 at outlet end 32 for supporting material 2 while still allowing the process fluid to pass through cartridge 16.

Reinforced wall 22 of cartridge 16 is designed to withstand a clamping force between feed section 12 and effluent section 14, described below. In order to ensure that the process fluid does not leak from processing device 10, cartridge 16 is tightly clamped between feed section 12 and effluent section 14. Wall 22 of cartridge 16 should be rigid enough to withstand the clamping force so that cartridge 16 does not collapse.

Support member 38 is preferably a sintered metal, such as Hastelloy, but can be any material that is permeable to the process fluids flowing through cartridge 16 and that is sufficiently strong to support material 2. Other possible materials of support member 38 include glass, sintered glass, Raney metals, electro-bonded membranes, etched alloy membranes, and fine meshed screens with gaps smaller than the minimum size of material 2, but large enough to allow the process fluid to flow adequately. In one embodiment, cartridge 16 can hold between about 0.1 cm³ to about 10 cm³, and preferably about 3 cm³ to about 5 cm³ of material 2.

The volumes provided above for processing device 10 are not limiting and are provided simply for context in the preferred case of a laboratory-scale device. It is conceivable that processing device 10 can be scaled up to a pilot plant or even a commercial scale or scaled down to micro-scale without varying from the scope of the present invention.

Returning to FIG. 1, feed section 12 provides a means to feed the process fluid to cartridge 16 placed in cartridge station 18 of processing device 10. Feed section 12 can be located above cartridge 16, as shown in FIG. 1, or below cartridge 16 so that the process fluid can flow either downward or upward through cartridge 16, depending on the application.

In one embodiment, feed section 12 includes a feed channel 40 having an exit end 42 with a generally outwardly extending flange 44 at an exit end 42. Flange 44 encircles a feed port 46 at exit end 42, through which the process fluid flows before passing into cartridge 16. The feed line 20 feeds a process fluid into feed channel 40. Preferably, feed line 20 is made from a flexible material, such as plastic or rubber, to allow for easy movement of feed section 12 while still avoiding breaking or detachment of feed line 20.

Inlet rim 34 of cartridge 16 abuts against flange 44 at exit end 42 of feed section so that inlet rim 34 of cartridge 16 surrounds feed port 46. In one embodiment, feed section 12 includes a seal between feed section 12 and cartridge 16 for sealing between feed port 46 and inlet end 30 of cartridge 16. The seal provides for parallel sealing to effectively seal between cartridge 16 and feed section 12. In one embodiment, shown in FIG. 1, the seal is an annular insulating pad 50 that is retained at flange 44 so that inlet rim 34 of cartridge 16 abuts feed insulating pad 50 to seal and insulate cartridge 16 from its surroundings. Alternatively (not shown), feed insulating pad 50 can be located on the inlet rim of the cartridge to seal between cartridge 16 and feed section 12. Feed insulating pad 50 is made of a thermally resistant and stable material, such as a polymer capable of withstanding the highest expected temperature of processing device 10.

Continuing with FIG. 1, feed section 12 can also include a heater 52 surrounding feed channel 40 to heat the process fluid as it flows through feed channel 40. Examples of heater 52 include a heating jacket using a heating fluid or an electrical heating element. Although for most applications it is desirable to heat the process fluid before it enters cartridge 16, for some applications it may be desired to cool the process fluid first. Therefore, heater 52 can also be a cooler and would still fall within the scope of the present invention.

Effluent section 14 provides a path for the process fluid to exit cartridge 16 after the fluid has contacted material 2. Effluent section 14 includes an entrance end 54 having an effluent port 56, an exit 58, and a conduit 60 extending through effluent section 14 between effluent port 56 and exit 58. The process fluid flows out of cartridge 16 and into conduit 60 through effluent port 56, the process fluid can then flow through conduit 60 and out of effluent section 14 through exit 58. An effluent line 62 provides a path for the process fluid to flow away from effluent section 14. Preferably, effluent line 62 is flexible to allow for movement of effluent section 14 while preventing the breaking or detachment of effluent line 62. Like feed section 12, effluent section 14 can also be located either above or below cartridge 16, depending on the desired flow direction of the process fluid.

Continuing with FIG. 1, effluent section 14 may also include a cooler 108 for cooling the product gas within conduit 60. An example of a cooler 108 is a heat exchanger using cooling water, or some other cooling fluid, to cool the process fluid. Although for most applications it is desired that the process fluid be cooled after flowing out of cartridge 16, there are some applications where it may be desired to heat the process fluid within effluent section 14. Therefore, cooler 108 can also be a heater, such as a heating jacket or a heating element, and would not vary from the scope of the present invention.

Outlet rim 36 of cartridge 16 abuts entrance end 54 of effluent section 14 so that cartridge 16 is clamped between effluent section 14 and feed section 12. Outlet rim 36 of cartridge 16 surrounds effluent port 56 so that the process fluid flows into conduit 60. Entrance end 54 may also include an effluent seal for sealing between effluent port 56 of effluent section 14 and outlet end 32 of cartridge 16.

In one embodiment, shown in FIG. 1, the effluent seal is an effluent insulating pad 66 that is used to seal between cartridge 16 and effluent section 14 and to insulate cartridge 16 from its surroundings. Preferably, effluent insulating pad 66 is annular in shape and is retained at entrance end 54 of effluent section 14 so that outlet rim 36 of cartridge 16 abuts effluent insulating pad 66 to seal and insulate cartridge 16 from its surroundings. Effluent insulating pad 66 can also be retained at outlet rim 36 of cartridge 16 seal between cartridge 16 and effluent section 14.

As with feed insulating pad 50, effluent insulating pad 66 is preferably made of a thermally resistant and stable material, such as a polymer capable of withstanding the highest expected temperature of processing device 10. Although insulating pads 50 and 66 provide a simple means for sealing and insulating cartridge 16, other seals, such as the embodiment described below, can be used without varying from the scope of the present invention.

In one method, a seal is formed between cartridge inlet end 30 and feed port 46 and between cartridge outlet end 32 and effluent port 56 by clamping cartridge 16 between feed section 12 and effluent section 14. In order to clamp cartridge 16 between feed section 12 and effluent section 14, processing device 10 can include an actuator, such as rack-and-pinion system 112 described below, for raising and lowering either feed section 12 or effluent section 14. In one embodiment, shown in FIG. 1 and described below, the actuator is provided to move effluent section 14 upward and downward to clamp cartridge 16 against feed section 12. In an alternative embodiment (not shown) the actuator can be provided to move feed section 12 upward and downward so that cartridge 16 is clamped against effluent section 14. In another alternative (also not shown), both feed section 12 and effluent section 14 can each have an actuator to move both sections upward or downward to clamp cartridge 16 between feed section 12 and effluent section 14.

In a preferred embodiment, shown in FIG. 1, the actuator is a rack-and-pinion system 112. Rack-and-pinion system 112 includes a set of gears 114 separate from effluent section 14 having cogs 116 and for each gear 114 a rack 118 connected to effluent section 14, wherein racks 118 include cogs 120 that engage cogs 116. Rotation of gears 114 moves effluent section 14 up or down via racks 118. Although a rack-and-pinion system 112 is described as the actuator, other systems, such as hydraulic or magnetic systems, can be substituted for rack-and-pinion system 112.

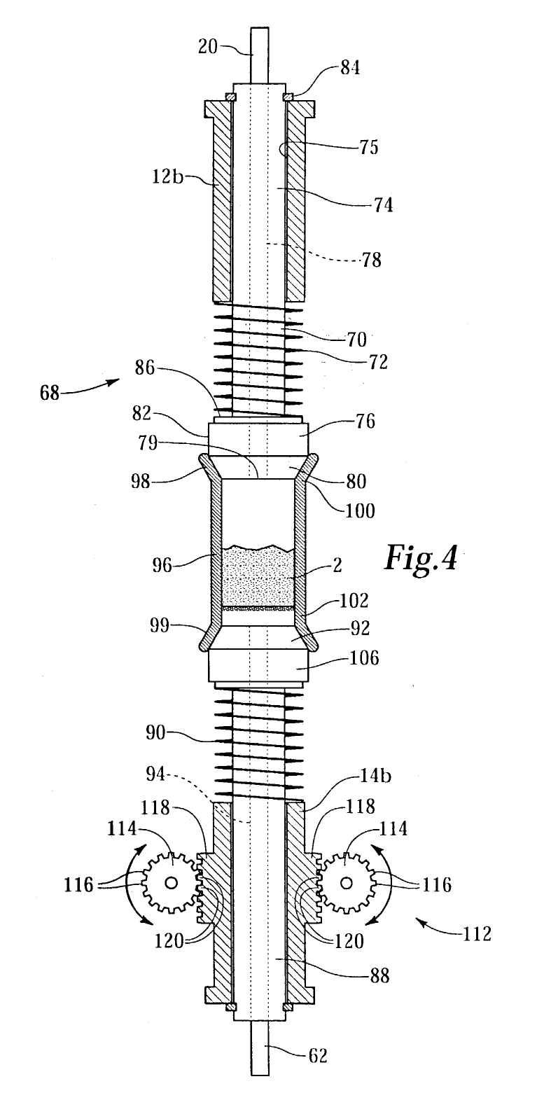

In an alternative embodiment of the present invention, shown in FIG. 4, a metal-to-metal sealing apparatus 68 is employed to seal cartridge 96 between feed port 46 and effluent port 56. Metal-to-metal sealing apparatus 68 is integrated with feed section 12b and effluent section 14b, and includes an insert 70 and a spring 72.

Insert 70 includes a generally cylindrical main section 74 and a sealing head 76 having a cylindrical outer surface 82 defining a diameter that is larger than the diameter of main section 74. Insert 70 defines an internal conduit 78 running throughout the length of insert 70, wherein the process fluid flows through conduit 78. Sealing head 76 ends in a truncated cone 80 which angles inwardly from outer surface 82 toward feed port 79 at the end of conduit 78. Insert 70 extends through a cylindrical bore 75 defined by the thickness of feed section 12b for translational movement of insert 70 therein. Main section 74 of insert 70 extends above feed section 12b and is engaged by a snap ring 84 which prevents withdrawal of insert 70 from the bottom of feed section 12b. Feed line 20 is connected to insert 70 and feeds the process fluid into conduit 78, as shown in FIG. 4.

Sealing head 76 provides a shoulder 86 that retains spring 72 between feed section 12b and sealing head 76 so that spring 72 acts to bias insert 70 away from feed section 12b and toward cartridge 96. A similar, but inverted insert 88 with a spring 90 and a truncated cone 92 is integrated with effluent section 14b to seal between effluent section 14b and cartridge 96 and allow the process fluid to flow from cartridge 96 into conduit 94 and out of effluent section 14b, as shown in FIG. 4.

In the alternative embodiment, alternative cartridge 96 differs from cartridge 16 of FIG. 1 in that each alternative cartridge 96 also includes frusto-conical section 98 at its inlet end 100 and frusto-conical section 99 at its outlet end 102 which are adapted to cooperate with truncated cones 80, 92 of inserts 70, 88 at inlet end 100 and outlet end 102 of cartridge 96 to provide a metal-to-metal seal between cartridge 96 and inserts 70, 88.

When cartridge 96 is placed between effluent section 14b and feed section 12b it is aligned so that frusto-conical sections 98, 99 of cartridge 96 line up with truncated cone 80 of feed section insert 70 and truncated cone 92 of effluent section insert 88. Effluent section 14b or feed section 12b is moved by rack-and-pinion system 112 (shown for moving effluent section 14b in FIG. 4) or another equivalent actuator so that cartridge 96 is clamped between feed section insert 70 and effluent section insert 88.

In the embodiment shown in FIG. 4, when effluent section 14b has been raised to a predetermined level, feed section spring 72 is compressed between feed section 12b and sealing section 76 of feed section insert 70. Similarly, effluent section spring 90 is compressed between effluent section 14b and sealing section 106 of effluent section insert 88.

Springs 72, 90 provide a force to bias feed section insert 70 tight against frusto-conical section 98 of cartridge 96 and to bias effluent section insert 88 tight against frusto-conical section 99. Springs 72, 90 ensure a tight metal-to-metal seal between inserts 70, 88 and cartridge 96. An advantage of metal-to-metal sealing apparatus 68 is that it can withstand much higher temperatures then traditional elastomer O-ring seals such as VITON™ or TEFLON™.

Returning to FIG. 1. during operation of processing device 10, cartridge 16 is moved into cartridge station 18 so that it is positioned between feed port 46 and effluent port 56. When inlet rim 34 and outlet rim 36 of cartridge 16 are in proper alignment with exit end 42 of feed section 12 and entrance end 54 of effluent section 14, rack-and-pinion system 112 raises effluent section 14. Effluent section 14 is raised until cartridge 16 is tightly clamped between feed section 12 and effluent section 14. After cartridge 16 is securely clamped between feed section 12 and effluent section 14 so that cartridge 16 is sealed between feed port 46 and effluent port 56, the process fluid is flowed through feed line 20 into feed section 12, where it may be heated by heater 52. The process fluid then flows through feed port 46 and into cartridge 16, where it processes material 2. After material 2 has been processed, the process fluid flows out of cartridge 16 and through effluent port 56 in effluent section 14 into conduit 60, where the process fluid may be cooled by cooler 108. After flowing through conduit 60, process fluid flows out of effluent section 14 through exit 58 and into effluent line 62.

When material 2 has been sufficiently processed, flow of the process fluid is stopped through processing device 10, and gears 114 are rotated so that effluent section 14 is lowered, unclamping cartridge 16 and releasing it from between feed port 46 and effluent port 56. After cartridge 16 is unclamped, cartridge 16 is moved away from cartridge station 18 between feed section 12 and effluent section. A second cartridge can move into cartridge station 18 to repeat the process for a different material 2 or with a different process step.

Preferably, a plurality of cartridges 16 is used with processing device 10 so that a plurality of materials 2 may be screened in a relatively short period of time. Each cartridge 16 of the plurality can contain the same material 2 to be processed, or each cartridge 16 can contain a different material 2 to be processed. Still more preferably, a plurality of cartridges 16 is used in an arrangement allowing automatic and controlled movement of each cartridge 16 into and out of cartridge station 18 of processing device 10.

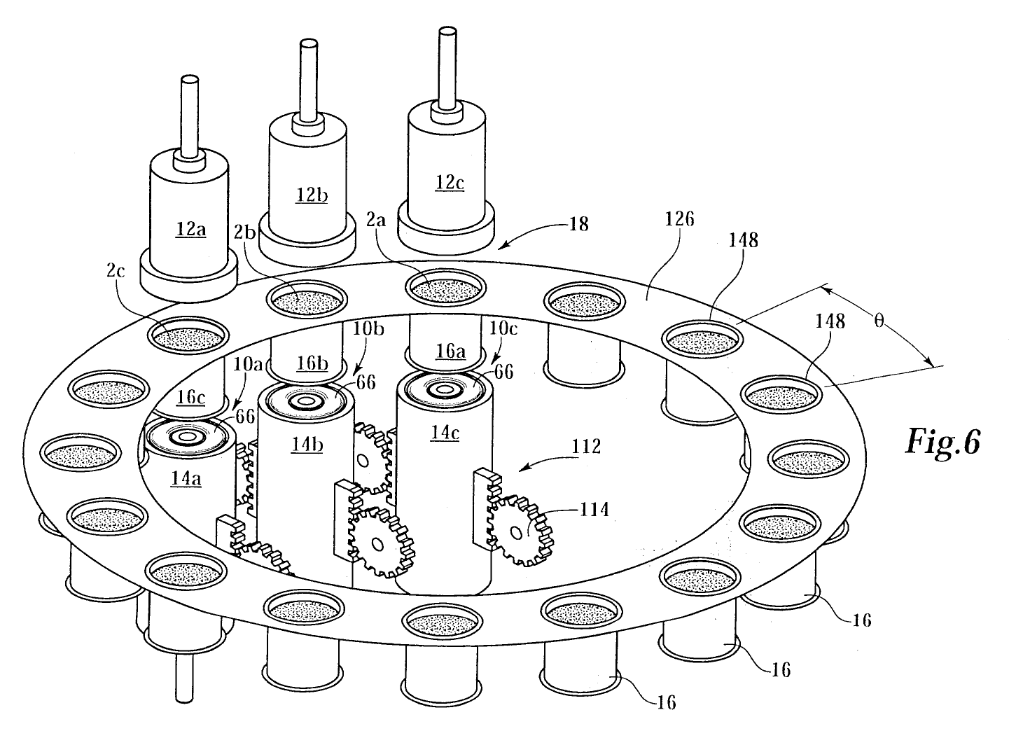

In order to easily manipulate a plurality of cartridges 16, a cartridge support is used to hold and support the plurality of cartridges 16. The cartridge support can be one of several embodiments for supporting a plurality of cartridges 16. For example, the cartridge support can be a cartridge belt 124 (FIG. 1), a carousel 126 (FIG. 6), or a cartridge tray 128 (FIG. 7). The cartridge support is capable of quickly and easily moving a plurality of cartridges 16 between feed section 12 and effluent section 14 of processing device 10.

Turning to FIGS. 1 and 5, cartridge belt 124 is designed to hold a predetermined number of cartridges 16 arranged in a row so that each cartridge 16 can be automatically positioned into and out of cartridge station 18 in a sequential order. Preferably, each cartridge 16 supported by cartridge belt 124 is spaced from its neighboring cartridges 16 by a predetermined distance D, so that a first cartridge 16a is spaced from a second cartridge 16b by the distance D, and from a third cartridge 16c by a distance equal to twice D. When a first cartridge 16a is to be moved out of cartridge station 18a and a second cartridge 16b is to be moved into cartridge station 18a then cartridge belt 124 is moved a distance equal to D. Equal spaced distancing between cartridges 16 is preferable because it allows for automated movement of cartridge belt 124 so that the method of processing materials 2 in cartridges 16 can be carried out automatically and efficiently.

Preferably, the movement of the cartridge support is automated and computer controlled by a cartridge position controller 130 to allow for automated and rapid performance of processing devices 10 for the processing of materials 2. Several cartridge position controllers 130 for moving cartridges 16 are known in the sample changing art. Cartridge position controller 130 may comprise any mechanical device with a suitably-controlled actuation system to position a selected cartridge 16. Cartridge position controller 130 preferably acts on cartridges 16 to position them in the location of cartridge station 18 and remove cartridges 16 from cartridge station 18. Alternately, cartridge position controller 130 can act on processing device 10 to position cartridge station 18 by locating processing device 10 in alignment with a selected cartridge 16.

Turning to FIG. 1, a simple cartridge position controller 130 is shown for positioning a linear array of cartridges 16 that extend along cartridge belt 124 for maintaining a distance D between adjacent cartridges 16. Upper rail 132 and lower rail 134 located on each end of the cartridges (shown only as being behind cartridges 16 in FIG. 1) serve as supports and guides to maintain vertical and horizontal cartridge position while a semi-rigid band 136 engages the end of cartridge belt 124 for linear translation of cartridges 16. Semi-rigid band 136 winds and unwinds from a reel 138 and a stepper motor 140 imparts controlled angular displacement to reel 138 that in turn advances or retards the linear positioning of cartridges 16 for their selective engagement with processing device 10. In such an arrangement the rails, reel and stepper motor may maintain a fixed relationship with the processing device through direct incorporation therewith.

An alternative arrangement, shown in FIG. 5, includes a plurality of processing devices 10a, 10b, 10c, 10d wherein each processing device 10a, 10b, 10c, 10d can perform a distinct process step. A processing device 10b is spaced from adjacent processing devices 10a, 10c by the same distance D as the space between adjacent cartridges 16a, 16b, 16c, 16d in cartridge belt 124. The plurality of processing devices 10a, 10b, 10c, 10d are arranged in an assembly line fashion so that a cartridge 16a can move between each of the plurality of processing devices in a predetermined order. For example, a first processing device 10a can perform a first process step, a second processing device 10b, adjacent to first processing device 10a, can perform a second process step, and a third processing device 10c, adjacent to second processing device 10b, performs a third process step, etc.

A first cartridge 16a can be positioned into cartridge station 18a between first feed port 46a and second feed port 56a of first processing device 10a, where first cartridge 16a is clamped between feed section 12a and effluent section 14a by an actuator to seal cartridge 16a between feed port 46a and effluent port 56a. After first cartridge 16a is clamped and sealed between feed section 12a and effluent section 14a, the first process step is performed on a material 2a in first cartridge 16a. Once the first process step is completed, first cartridge 16a is unclamped from between feed section 12a and effluent section 14a and first cartridge 16a is moved a distance D by cartridge position controller 130 via cartridge belt 124 to cartridge station 18b between feed port 46b and effluent port 56b of second processing device 10b, while a second cartridge 16b is moved into first cartridge station 18a.

Once first cartridge 16a is located in second cartridge station 18b and second cartridge 16b is located in first cartridge station 18a, first cartridge 16a is clamped between feed section 12b and effluent section 14b, and second cartridge 16b is clamped between feed section 12a and effluent section 14a. A second process step is performed on material 2a in first cartridge 16a and the first process step is performed on a material 2b in second cartridge 16b. This process can be repeated for additional process steps at a third processing device 10c, a fourth processing device 10d, etc. The multiple process steps can be performed on material 2a in first cartridge 16a, material 2b in second cartridge 16b, a third material 2c in a third cartridge 16c, a fourth material 2d in a fourth cartridge 16d, etc.

Moving on to FIG. 6, carousel 126 holds a plurality of cartridges 16 in a circular arrangement. Cartridges 16 are placed into cartridge holes 148 in carousel 126 so that each cartridge 16 is spaced from its neighboring cartridges 16 by an angle θ. In one embodiment, θ is defined as:

$$θ = \frac{{360}^{°}}{N}$$

where θ has units of degrees and N is the total number of cartridges holes 148 in carousel. When a first cartridge 16a is desired to be moved out of cartridge station 18 and a second cartridge 16b is desired to be moved into cartridge station 18, carousel 126 is rotated by θ degrees.

Carousel 126 also allows a plurality of processing devices 10a, 10b, 10c that are also arranged in a circular arrangement, as shown in FIG. 6, to be used so that each cartridge 16 in carousel 126 may have a corresponding processing device 10 to perform a specific process step. The arrangement shown in FIG. 6 allows for each processing device of the plurality of processing devices 10a, 10b, 10c to perform a distinct process step. This arrangement allows cartridges 16a, 16b, 16c in carousel 126 to undergo a prescribed series of process steps. For example, a first processing device 10a can perform a first process step, which can be a material preparation step, a second processing device 10b can perform a second process step, such as a heat treatment step, and a third processing device 10c can perform a third process step, such as a finishing step. A material 2a in a first cartridge 16a can undergo a first process step at first processing device 10a. After the first process step is completed, carousel 126 can be rotated by θ degrees, moving first cartridge 16a from first processing device 10a to second processing device 10b and moving a second cartridge 16b into first processing device 10a. A second process step can be performed on material 2a in first cartridge 16a while the first process step can be simultaneously performed on a material 2b in second cartridge 16b. After first material 2a has completed the second processing step and second material 2b has completed the first processing step, carousel 126 can be rotated by θ degrees so that first cartridge 16a is moved from second processing device 10b to a third processing device 10c, second cartridge 16b is moved from first processing device 10a to second processing device 10b, and a third cartridge 16c can be moved into first processing device 10a so that a third process step can be performed on material 2a in first cartridge 16a, the second process step can be performed on material 2b in second cartridge 16b and the first process step can be performed on a material 2c in third cartridge 16c.

As shown in FIG. 7, another embodiment of the cartridge support comprises a cartridge tray 128 for holding a plurality of cartridges 16 in a grid arrangement with a predetermined number of rows and a predetermined number of columns. Cartridge tray 128 may be configured to hold any number of cartridges 16. For example, cartridge tray 128 can hold 6, 8, 12, 24, 48, 96 or 384 of cartridges 16. In one embodiment, shown in FIG. 7, cartridge tray 128 is arranged in a configuration having eight rows along an X-direction and six columns along a Y-direction so that cartridge tray 128 can hold a total of forty-eight cartridges 16. Also, the full capacity of cartridge tray 128 need not be used. For example, a cartridge tray 128 designed to hold up to forty-eight cartridges 16 may be used to support only twenty-four. Processing device 10 is flexible in this respect, because the number of cartridges 16 being used by processing device 10 can be changed simply by adding or taking away a desired number of cartridges 16 from cartridge tray 128.

Cartridge tray 128 allows for two dimensional manipulations of cartridges 16 in both the X direction and the Y direction, as shown in FIG. 7. Manipulation in both the X direction and the Y direction allows many cartridges 16, and thus many samples of material 2, to be moved in a relatively small space. For example, if for a certain application a first cartridge 16a located in the first row and the third column can be moved out of cartridge station 18 and any one of the other forty-seven cartridges 16 can be moved into cartridge station 18, such as second cartridge 16b in the fifth row and the second column or a third cartridge 16c in the eighth row and the fourth column.

In another embodiment of the present invention, one or more sets 150 of processing devices 10 are provided, wherein each set 150 of processing devices 10 corresponds to all of the cartridges 16 in a particular row of cartridge tray 128. Each set 150 of processing devices 10 performs one or more process steps on materials 2 in the cartridges 16 in a corresponding row. Each processing device 10 of a set 150 can perform the same process step, such as an oxidizing heat treatment step, each processing device 10 can perform a different process step, or banks of more than one processing device 10 can perform different process steps with each processing device 10 in a bank performing the same process step.

The embodiment in FIG. 7 is flexible in this respect, because it allows a plurality of materials 2 to be placed in the plurality of cartridges 16, which can be arranged in cartridge tray 128 in any one of several configurations, including the following three examples. First, a different material 2 placed in each cartridge 16 in cartridge tray 128 so that there are a total of forty-eight materials 2 being processed. Second, a different material 2 in each row of cartridge tray 128 so there are a total of eight materials 2, wherein each material 2 undergoes all of the process steps associated with each set 150. Third, a different material 2 in each column of cartridge tray 128 so that there are a total of six materials 2 wherein each material 2 undergoes only one of the process steps associated with one of the processing devices 10 of each set 150.

Preferably any apparatus or method of this invention will retain cartridges 16 in a retaining device such as carousel 126, cartridge tray 128, or cartridge belt 124 and rails 132,134 in manner that permits translation movement of the cartridges within the retaining device. This permits the sealing of the cartridge on both ends to occur readily by only applying pressure to one side of the device through movement of only the feed section 12 or outlet section 14 of the processing device 10.

This preference for cartridge movement within the retaining device does not exclude the use of cartridge, retaining device, and processing device arrangements that achieve sealing in a different manner. For example, a fixed cartridge arrangement may rigidly retain cartridges within the retaining device or even have cartridges formed unitarily as part of the retaining device. Such a fixed cartridge arrangements may have enough inherent flexibility for sealing at both ends by the application of movement to only an inlet of section 12 or outlet of section 14. Alternately sealing of the cartridge elements with movement from only one of the inlet or outlet sections may occur by allowing translation of the entire retaining device assembly. In fact, the use of one or more expanding seal elements may obviate the need for any movement of the inlet and outlet sections. Possible arrangements for expanding seals may interpose an expandable element between the seal element and the seal seat of the inlet or outlet section or provide inflation of the seal itself.

Although only three configurations are described above as examples, one having ordinary skill in the art would recognize that any one of several other configurations can be used without varying from the scope of the present invention.

Processing device 10 processes materials 2 using a method that includes the steps of loading material 2 to be processed into a cartridge 16, wherein cartridge 16 includes an inlet, for example at inlet end 30, and an outlet, for example at outlet end 32, positioning cartridge 16 between feed port 46 and effluent port 56, sealing between feed port 46 and cartridge inlet end 30 and between effluent port 56 and cartridge outlet end 32, flowing a fluid through cartridge 16, and releasing cartridge 16 from between feed port 46 and effluent port 56.

Another method includes providing a cartridge support for holding one or more cartridges 16, such as cartridge belt 124, carousel 126 or cartridge tray 128, wherein the positioning step describe above includes moving the cartridge support so that cartridge 16 is positioned between feed port 46 and effluent port 56. The method can also include the steps of clamping the cartridge between feed port 46 and effluent port 56 wherein the sealing step includes the clamping, heating the fluid before flowing the fluid through cartridge 16, and cooling the fluid after flowing the fluid through cartridge 16.

Yet another method includes the steps of removing cartridge 16a from cartridge station 18a, see FIG. 5, positioning cartridge 16a between a second feed port 46b and a second effluent port 56b, sealing between second feed port 46b and cartridge inlet end 30a and between second effluent port 56b and cartridge outlet end 32a, flowing fluid through cartridge 16a, and releasing cartridge 16a from between second feed port 46b and second effluent port 56b.

1. An apparatus for contacting fluid with a plurality of solid material samples in a series of cartridges, comprising:
a processing device for passing fluid through an interior volume of a cartridge, the process device having a feed port and an effluent port, the cartridge having an inlet and an outlet;
at least one cartridge position controller for positioning the cartridge between the feed port and the effluent port and for removing the cartridge from between the feed port and the effluent port;
a first seal for sealing between the feed port and the cartridge inlet; and
a second seal for sealing between the effluent port and the cartridge outlet.

2. An apparatus according to claim 1, further comprising a feed section and an effluent section, wherein the feed port is in the feed section and the effluent port is in the effluent section.

3. An apparatus according to claim 2, further comprising an actuator for moving at least one of the feed section and the effluent section to clamp the cartridge between the feed section and the effluent section to seal between the feed port and the cartridge inlet and between the effluent port and the cartridge outlet.

4. An apparatus according to claim 3, wherein the actuator is a rack-and-pinion system.

5. An apparatus according to claim 1, further comprising a cartridge support for retaining the cartridges, wherein the cartridge position controller movies the cartridge support to position a selected cartridge between the feed port and the feed port.

6. An apparatus according to claim 5, wherein the cartridge support positions the cartridges within a plane of principle movement and the cartridge support retains the cartridges in a manner that permits translation of the cartridges perpendicular to the plane of principle movement.

7. An apparatus according to claim 1, wherein each cartridge includes a porous support within the interior volume of the cartridge for supporting material while allowing fluid to pass.

8. An apparatus according to claim 1, wherein the cartridge has a first rim at the inlet and a second rim at the outlet, wherein the first rim abuts the feed port and the second rim abuts the effluent port.

9. An apparatus according to claim 1, further comprising a heater for heating fluid.

10. An apparatus according to claim 1, further comprising a cooler for cooling fluid.

11. An apparatus according to claim 1, further comprising a cartridge belt for holding the cartridge, wherein the cartridge position controller moves the cartridge belt to position the cartridge between the feed port and the effluent port.

12. An apparatus according to claim 1, further comprising a tray for holding the cartridge, wherein the cartridge position controller moves the tray to position the cartridge between the feed port and the effluent port.

13. An apparatus according to claim 1, further comprising a carousel for holding the cartridge, wherein the cartridge position controller moves the carousel to position the cartridge between the feed port and the effluent port.

14. An apparatus according to claim 1, wherein at least one of the first seal and second seal is a pad for sealing respectively between the feed port and the cartridge inlet and the effluent port and the cartridge outlet.

15. An apparatus according to claim 1, wherein at least one of the first seal and the second seal is expandable.

16. A method for processing material, comprising the steps of:
loading material to be processed into a cartridge, wherein the cartridge includes an inlet and an outlet;
positioning the cartridge between a feed port and an effluent port using a cartridge position controller;
sealing between the cartridge inlet and the feed port using a seal and between the cartridge outlet and the effluent port using a seal;
flowing fluid through the cartridge;
releasing the cartridge from between the feed port and the effluent port using the cartridge position controller.

17. A method according to claim 16, wherein the cartridge position controller is a cartridge belt for holding the cartridge, wherein the positioning step includes moving the cartridge belt so that the cartridge is positioned between the feed port and the effluent port.

18. A method according to claim 16, wherein the cartridge position controller is a tray for holding the cartridge, wherein the positioning step includes moving the tray so that the cartridge is positioned between the feed port and the effluent port.

19. A method according to claim 16, wherein the cartridge position controller is a carousel for holding the cartridge, wherein the positioning step includes moving the carousel so that the cartridge is positioned between the feed port and the effluent port.

20. A method according to claim 16, further comprising heating the fluid.

21. A method according to claim 16, further comprising cooling the fluid.

22. A method according to claim 16, wherein the sealing step includes clamping the cartridge between the feed port and the effluent port.

23. A method according to claim 16, further comprising removing the cartridge from between the feed port and the effluent port using the cartridge position controller, positioning the cartridge between a second feed port and a second effluent port using the cartridge position controller, sealing between the cartridge inlet and the second feed port and between the cartridge outlet and the second effluent port, flowing fluid through the cartridge and releasing the cartridge from between the second feed port and the second effluent port using the cartridge position controller.

24. A method according to claim 23, wherein sealing between the second feed port and the cartridge inlet and between the second effluent port and the cartridge outlet includes clamping the cartridge between the second feed port and the second effluent port.

25. A method according to claim 16, further comprising providing a cartridge support for holding the cartridges, wherein the positioning step includes moving the cartridge support so that a cartridge is positioned between the feed port and the effluent port.
